(19) United States
(12) Patent Application Publication
(10) Pub. No.: US 2005/0227338 A1
(43) Pub. Date: Oct. 13, 2005

(54) Production of biomass and single cell protein from industrial waste oils, oily sludge from ships and other sources of oily wastes

(76) Inventors: Satyanarayana Ganti, Plainsboro, NJ (US); Dinkar Ganti, (US)

Correspondence Address: LAW OFFICES OF NANCY A. BIRD, 231 WALTON AVENUE, SO. ORANGE, NJ, 07079, US

(21) Appl. No.: 11/089,092

(22) Filed: Mar. 24, 2005

Related U.S. Application Data
(60) Provisional application No. 60/556,387, filed on Mar. 25, 2004.

(51) Int. Cl. C12P 7/64; C12N 1/21

(52) U.S. Cl. 435/134; 435/252.3

(57) ABSTRACT

High yield low cost processes for generating nutrient rich biomass, including SCP, and ECP containing biosurfactants; from waste oils, including oily sludge from ships, without added nutrients, and improved separation of oil from biomass.

### CROSS-REFERENCE TO RELATED APPLICATIONS

This application claims priority from Provisional Application 60/556,387 filed Mar. 24, 2004.

### FIELD OF THE INVENTION

The present invention relates to processes for the production of biomass, and bioremediation of oil spills; using a bio-dispersion process, which yields nutrients, including Single Cell Proteins; and bio-surfactants.

### BACKGROUND OF THE INVENTION

A method of bioremediation of oils spills is disclosed in U.S. Pat. No. 6,267,888. Though this method has been successful in cleaning up oil spills, sources of waste oil such as the bilge water of ships, it produces no waste product, just clean water, and nutrient rich biomass.

Methods of producing protein from hydrocarbons have been limited by the cost of expensive purified hydrocarbons, believed to be the necessary starting materials, not waste oil, if food protein was to be produced. In addition, the production of protein from hydrocarbon has received little attention because of the high costs of the production process, stringent in-house conditions, costly nutrient formulations, need for prevention of incidental contaminations during fermentations, low volume yields of biomass, difficult recovery process and costly effluent discharge practices.

Use of biomass for the production of single cell protein has been practiced for centuries, as seen in the case of yeast production. In fact, both bacteria and yeasts have been used in the past as food because of their fast rate of growth. According to Bunker (1968), Escherichia coli has a specific growth of 59.7/day while yeast Hansenula anomala has 13.8 per day. [Bunker, H. J. “Sources of single-cell protein: Perspective and prospect.” In Single Cell Protein Ed. Mateles & Tannenbaum, M.I.T Press, Cambridge Mass., pp 67-77; 1968) Yeast produce protein at a very fast rate. For example, a steer weighing more than 1000 lbs synthesizes only 1 pound of protein a day whereas yeast cells of the same weight produce more than 50 tons of protein; an efficiency of 112,000 times that of steer. However, there are many obstacles, including cost, which have prevented the use of yeast, or bacterial growth protein as a nutritional supplement.

Producing proteins from hydrocarbons has been described in detail by J. B. Davis in his book on Petroleum Microbiology Davis (1967). [Davis, J. B. “Petroleum Microbiology” Elsevier Publishing Company, New York, 1967.] Yamada et al., compared total cell yield in flask cultures for bacterium Pseudomonas aeruginosa and yeast Candida tropicalis and reported that C. tropicalis produced 7.30 Gms per liter on normal hexadecane whereas P. aeruginosa produced less than 3.0 gms per liter on the same substrate. [Yamada, K; Takahashi, J; Kawabata, Y; Okada, T and Onihara, T “SCP from yeast and bacteria grown on hydrocarbons” in Single Cell Protein, Ed. by Mateles & Tannenbaum, M.I.T Press, Cambridge Massachusetts, pp:192-207, 1968.]

Earlier work in Taiwan (Ko & Yu, 1968) described a process for large-scale production of SCP using fuel oil as the hydrocarbon substrate and Pseudomonas species in a 50-kiloliter fermentor using both batch culture and continuous fermentation. [Ko, PC and Yu, Y “Production of SCP from hydrocarbons: Taiwan” in Single Cell Protein, Ed. by Mateles & Tannenbaum, M.I.T Press, Cambridge Massachusetts, pp: 255-262; 1968.] This suggests that such volumes of biomass can be produced and the present invention provides methods for such production from oily sludge.

It is an object of the present invention to provide a process for the production of the biomass while cleaning up oil from industrial waste oils, oily sludge from ships and other sources of oily waste.

It is a further object of the invention to provide a high-yield, low cost, process for the production of nutrient rich biomass, and bio-surfactants, from industrial waste oils, oily sludge from ships and other sources of oily waste. In a further object of the present invention, the process requires only medium to low speed centrifuges, and requires no detergents.

It is a further object of the present invention to provide a process for high yield production of biomass, and SCP, which process utilizes a self contained system which requires no continuing addition of nutrients to either the oil phase or the water phase.

It is a yet further object of the present invention to provide a process which provides extra-cellular polymeric substances (EPS), from the cell free extract separated from the whole cell biomass. The ECP produced may be used as a bio-surfactants, or source of nutritional supplements.

It is another object of the present invention to provide a simplified process for making nutritional supplements from ECP, which do not contain any endotoxin (lipopolysaccarides), and can be considered safe for any nutritive addition product for animals and humans.

### BRIEF SUMMARY OF THE INVENTION

These objects, as well as other objects which will become apparent from the discussion that follows, are achieved, in accordance with the present invention, which comprises a high yield low cost processes for generating nutrient rich biomass and bio-surfactants; from waste oils, including oily sludge from ships, without added nutrients, and improved separation of oil from biomass; and the nutritional products isolated from the biomass. The high yield low cost processes for generating nutrient rich biomass from waste oils may also be used to make bi-surfactants. In addition, the present invention encompasses the elimination of waste oils by an inexpensive process which results in biomass, carbon dioxide and water.

According to the methods of the present invention, nutritionally rich whole cell biomass may be produced from waste oil, by isolating a source of waste oil in a container, adding to the container an oleophilic suspension comprising physiologically active, species/strains of bacteria that are oil-consuming, in a fatty substance, comprising an oleophilic nutrient as a source of nitrogen and phosphorus for said bacteria, and excess water; agitating the mixture for a fixed period of time, to produce biomass. The hydrocarbon oil is transformed into carbon dioxide, and water by the oil eating bacteria. These steps comprise the method of eliminating or reducing waste oil, which is particularly effective with eliminating oil that is difficult to recycle, yielding 90% elimination of such oils. Any residual oil remaining in the container may be removed by solvent extraction. Thereafter, the solvent may be recycled to be used again for solvent extraction, and the residual waste oils recycled into the container. It should be noted that the end products of this waste oil elimination process are biomass, which may be harvested, and has many uses, producing carbon dioxide, and nutrient rich water as by-products: and leaving no residue or other dangerous waste products for disposal.

To separate the whole cell biomass from the oil-free, water-based, their mixture may be centrifuged, requiring only low to medium speed, or no more than about 20,000 rpm. The whole cell biomass may be used to isolate protein, carbohydrates, amino acids, etc., which have use in nutritional supplements for animal and human consumption; as well as vitamins, antibiotics, specific bio-surfactants. The cell free extract is a useful bio-surfactant, and a source of nutritional supplement.

Examples of the oil consuming bacteria useful in the methods of the present invention are Pseudomonas aeruginosa, Phenylobacterium immobile, Stenotrophomonas maltophila, Gluconobacter cerius, Agrobacterium radiobacter and Citrobacter freundi. It is preferred to use a combination, or consortium of bacteria because of their differing abilities to consume various types of oils. However, when a particular product requires specific or individual bacteria, these may be used in the methods of the present invention. In addition, it should be noted that oil consuming yeasts and fungi may be substituted for the bacteria in practicing the methods of the present invention. The consortium of bacteria may be isolated using the biodispersion process described in U. S. Pat No. 6,267,888. After mixing the bacteria with the waste oil source and additional water, as required, the mixture may be agitated to ensure a supply of oxygen throughout the mixture, to support bacterial growth and multiplication resulting in oil consumption. There is no need to provide an additional source of oxygen, simple exposure to the atmosphere, as agitation of the mixture are sufficient to support the production of biomass. The process should be conducted at about 30 degrees C., which may require the addition of heat, depending on climate, and the conditions of the facility in which the process is undertaken.

A nutrient rich extra cellular polymeric substance may be produced from the mostly cell-free extract, by first filtrating through a 0.22 micron filter paper, and then precipitation with acetone, in a ratio of 3:1 acetone: cell free extract; or with ethyl alcohol, or other inorganic salts such as ammonium sulfates at the necessary proportions, and cooling the mixture to about 4 degrees C. for at least about 10 to 12 hours, and centrifuging the mixture at low to medium speed, or no more than 20,000 rpm to separate out the precipitated extra cellular polymeric substance from the eluent acetone, and water.

The extra cellular polymeric substance is a bio-surfactant, or an excellent source of nutritional supplements, including protein, carbohydrates, amino acids, etc.; as well as other valuable products such as vitamins, antibiotics and specific bio-surfactants. The extra cellular polymeric substance is also free of endotoxin Lipopolysaccarides, which may make it easier to isolate these nutrients. It is a white material, water soluble, and generally clear when in solution, and may therefore be easily incorporated into nutritional supplement products.

The method of producing whole cell biomass from hydrocarbon oils according to the present invention is more economical than previous methods because the starting product is simply waste oil, such as oily sludge from ships and not purified sources used by others, and does not require the addition of nutrients, other than those included with the bacteria consortium. None-the-less, the process of the present invention, using SpillRemed, can produce biomass comprising about 24% by weight carbohydrate and about 34% by weight protein, from simple sources of waste oil. The extra cellular polymeric substance extracted from the cell free extract comprises about 56% by weight carbohydrate and about 43% by weight protein. For a full understanding of the present invention, reference should now be made to the following detailed description of the preferred embodiments of the present invention.

### BRIEF SUMMARY OF THE DRAWINGS

FIG. 1 is a flow chart showing key stages in biomass production.

FIG. 2 is a schematic representation of the fermentor process.

FIG. 3 is a schematic representation of the open tank process.

### DETAILED DESCRIPTION OF THE INVENTION

The concept of using oily sludge for production of Single Cell Protein (SCP) is entirely novel. The methods of the present invention use the activities of microbial cells to transform waste sludge oil from, e.g., ships, into beneficial biomass. The biomass produced yields many nutrients, and, also, bio-surfactants. In the process, the oil is extracted from the water and biomass, before separating the whole cell biomass from the nutrient rich cell free extract.

Process Description

The present invention provides processes which may be practiced using simple bioreactors, in either a laboratory or in the field, to produce commercially viable biomass from waste industrial oil.

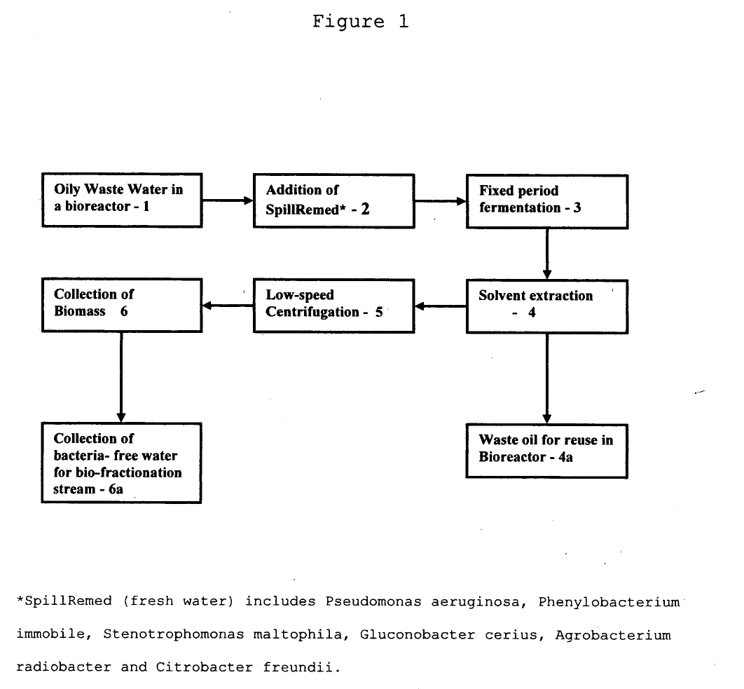

The flow chart in FIG. 1 shows some key steps in biomass production.

Important parameters for successful production of biomass have been listed by Bunker (1968) and are given below.
1. Rapid growth
2. Simple media
3. Suspended culture
4. Simple separation
5. Efficient utilization of energy
6. Disposable effluent
   Rapid Growth:

It is preferred to use mixed consortia of bacteria, such as that available in SpillRemed. The bacteria in SpillRemed, have demonstrated very active and rapid growth even under stress conditions in the remediation of oil spills. They have also not shown any selectivity in the nature of hydrocarbon source and hence may be used with a wide variety of waste hydrocarbons produced in the industry. The SpillRemed product is an oleophilic suspension comprising physiologically active, species/strains of bacteria that are oil-consuming, in a fatty substance, comprising an oleophilic nutrient as a source of nitrogen and phosphorus for said bacteria. By placing the bacteria in an oleophilic suspension; they become attracted to oil and may more easily cling to oil droplets. Also, including the nitrogen and phosphorus in the suspension means that nutrients do not have to be later added to the water, a cumbersome and inefficient method.

Growth on Simple Media:

The bacteria in SpillRemed require water for promoting remediation of waste oil. Water is the cheapest and simplest form of bulk ingredient in any media. Furthermore since addition of nutritive salts in water or oil phase are not required at any stage of the process, the overall process is cost effective.

Suspended Cells:

The bacterial cells in SpillRemed are free-living planktonic forms and are uniformly distributed in the medium during the process of bioremediation. It is therefore easier to separate these cells from the culture liquid in the bioreactor after solvent extraction of residual oil.

Ease of Separation

The planktonic bacterial cells are easily collected by low speed centrifugation. As the bacteria are grown on hydrocarbon substrate, the dispersed oil is removed through solvent extraction, permitting the water containing bacterial cells to be effectively centrifuged at low speed.

Discharge in the Waste Stream

At the end of the bioreactor, or fermentation process and solvent extraction the liquor remaining for disposal is nutrient rich water, which can be safely discharged in to the waste stream after determining if it meets the local environment standards. As there are no salts or nutrients added during the entire process, the discharge water does not require pre-treatment.

Cost Effectiveness

Waste oil is disposed of by the producer at a cost. Using the process of the present invention, the biomass produced may be sold commercially.

Significance:

Some animal feeds use growth hormones or other artificial additives that can have potential deleterious effects on the reproductive and endocrine systems of the farm animals, and they may also contribute to pollution of surface and subterranean water.

Bacterial biomass can provide a safe and reliable alternative to supplemental proteins currently used as animal feed.

In addition, the development of a cheap, safe and reliable alternative protein supply based on high quality bacterial biomass may become the worlds best solution to the need of a protein source in countries that are chronically in need for such a supply.

Optimization of Biomass Production

Studies have indicated a typical doubling time for oil-eating bacterial population is about 24 hours and at least a three-log doubling of total bacteria recorded during the exponential phase of growth at 25° C.

A. Temperature

Temperature, aeration and inoculum's size have a great influence on production of bacterial biomass, and need to be adjusted for the specific bacteria utilized in the process.

B. Aeration:

The oil-eating bacteria require oxygen because they are aerobic. Hydrocarbon assimilation by bacteria is an oxidative process and hence bacterial growth is highly dependent on a good supply of oxygen. However, atmospheric oxygen and agitation of the mixture are sufficient to meet the need of the bacteria. Oxygen is consumed by bacteria in reactions whereby hydrocarbons are first terminally oxidized, and subsequently pass through an alcohol intermediate. The resulting metabolic acids are then decarboxylated to provide energy to the cells, with the evolution of carbon dioxide.

C. Inoculum size:

One gallon of SpillRemed is effective in the bioremediation of 10 gallons of oil in field evaluations. Thus, the inoculum size should be 10% of the total volume of oily waste. Further, non-volatile and weathered oils are known to be more difficult to bioremediate than diesel and fuel oils. Hence the time required for complete bioremediation of sludge from ships along with biomass production may not follow the standard inoculum volumes and these values have to be determined beforehand, in relation to the time allotted for the process.

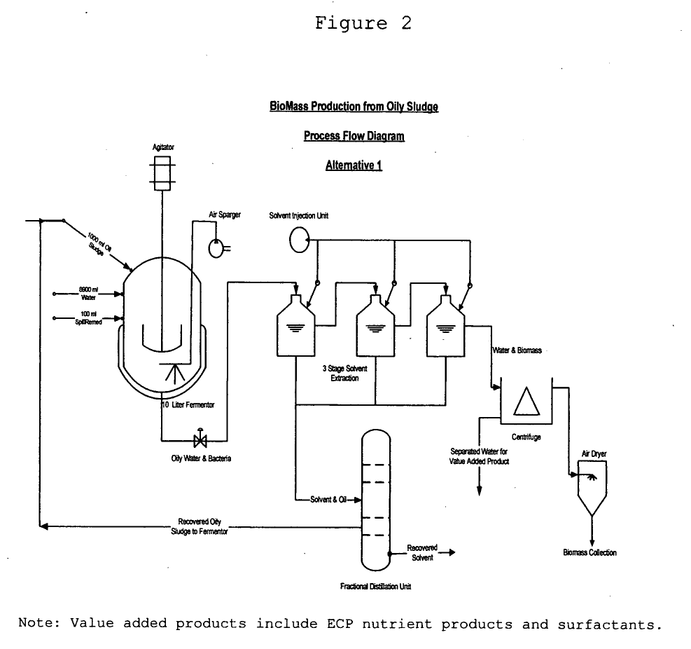

A schematic representation of the fermentor process is given in FIG. 2.

Open Tank Process:

The primary difference between the fermentor and the open tank process is the type of the container. The open tank is not a pressure vessel but is a simple drum equipped with a circulating pump for aeration and mixing the contents and heater to control the temperatures. The open tank process reduces the cost of capitalization during commercial production. Production costs will however be less in both the processes since there will not be any recurring costs of nutrient additions and will not need high capitalization costs.

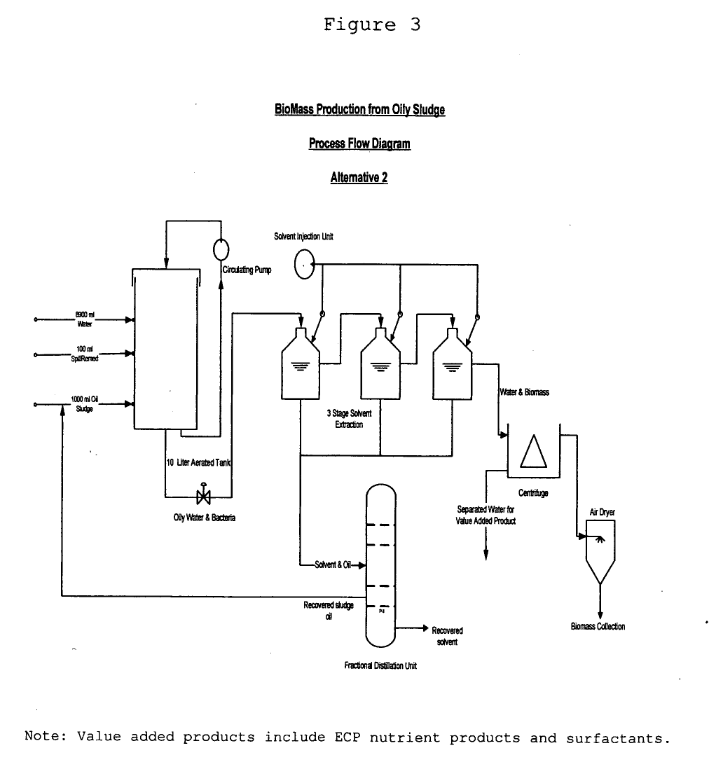

A schematic diagram for this process is given in FIG. 3.

### COMPARISION EXAMPLE

Seawater was pre-filtered and transferred in a 1.0 L jars with a three-way stopcock. It diesel oil pre-charged with Inipol nutrient was taken in the jar and the volume of oil was in the ratio of a 1.0 ml per 100 ml of seawater, or 10.0 ml for one liter of seawater. The medium thus prepared was then inoculated with 50.0 ml of a consortium of bacteria [Pseudomonas aeruginosa; Pseudomonas nautica; Vibrio natrigens; Micrococcus varians] grown for 24 hours. The experiment was terminated at the end of 120 hours. The dispersed oil was that extracted with analytical grade chloroform and the seawater was then centrifuged at 12,000 rpm, to remove all the suspended cells from the medium. The cells collected at the bottom of the centrifuge tubes were kept separately for further application.

The filtrate was further filtered through 0.22 μ membrane filter to remove any residual cells and the cell free filtrate was thus obtained. The cell free filtrate was then mixed with three volumes of chilled acetone to enable the precipitation of the extracellular metabolites produced by the consortium of bacteria. The flask was kept at −4° C. overnight for complete precipitation of the metabolic products. The filtrate with a precipitate was again centrifuged a 20,000 rpm to collect all the precipitate and the aqueous solvent was kept for reuse after distillation. The purified compound was then kept in dialysis bags or some desalting mechanism to remove interfering salts. The purified compound was then dried in an incubator at 40° C. for least 72 hours, or until the compound is dry. The purified compound was found to be a glycolipid consisting of proteins, carbohydrates, lipids and uronic acid as main constituents. The compound was found to be neutral in reaction indicating that the uronic acid recorded might be in a free state. A yield of 0.64 g for every liter was recorded when the experiments were terminated at the end of the 8th day, though optimum yield was recorded at the end of the 5^{th} day, and all experiments were terminated on the 5th day.

Properties of the compound are given below

| | |
|---|---|
| Polysaccharide's | 35-45% |
| Lipids | 25-40% |
| Protein | 6-10% |
| Uronic acid | 4-7% |

Prior Art Attempts at Biomass Recovery:

Wang had earlier described the process of cell recovery from hydrocarbons. [DIC “Cell Recovery” in Single Cell Protein, Ed. by Mateles & Tannenbaum, M.I.T Press, Cambridge Massachusetts, pp:217-228; (1968.)] The hydrocarbon feed used by Wang is purified normal alkane containing C₁₀ through C₂₀ n-paraffins. A typical range of concentration of the hydrocarbon in the feed would be, according to the author, 10 to 20 g/liter yielding an approximately equal concentration of cells. The broth containing the cells and unassimilated hydrocarbon is separated by centrifugation into three streams:
Cell paste
Aqueous phase
Hydrocarbon phase
   The entire process described by the Wang indicates a need for conserving the nutrients in the fermentation broth by recycling. Purification fermentors have been used for cleaning the bacterial cells. These cells were washed by incorporating surface active compounds.

Gutcho later described another process for recovery of cells after fermentation, which is also equally or more complex than that of Wang (1968). [S “Proteins from Hydrocarbons” Pub. Noyes Data Corporation, New Jersey, 144-216; (1973).] This involves removal of non-normal materials and impurities by filtration through molecular sieve and cleanup of desorbed hydrocarbons by passage through a silica gel column. The bacterial cells are then separated from the media by conventional centrifugation followed by steps of dewatering. Phase separation of hydrocarbons and bacteria associated with the oil phase can be aided by introduction of microfine bubbles. The floating mixture of hydrocarbons and bacterial cells can thus be separated from the lower water phase. The above process can be repeated to recover cells and hydrocarbons in amounts up to 99%.

Just before separation, the addition of an immiscible organic liquid of density greater than that of the aqueous phase, and which may be easily volatilized, can also improve 1) harvesting of bacteria from the aqueous medium; 2) recovering the cells from the organic phase by centrifugation; 3) subsequent evaporation of organic solvent and drying of the cells.

In some instances, it may be necessary to break the creamy emulsion containing microorganisms. This can be accomplished by the use of surface-active agents, e.g., alkylmonosulfonate and the methosulfonate of a quaternary fatty amine in amounts up to 0.15% relative to the total emulsion and mild elevation of heat to between 40° and 60° C.

Cells withdrawn from the fermentor can be acidified to pH 3.5 with Phosphoric acid prior to heat Pasteurized to render the bacteria non-viable. Partial sterilization at a temperature less than 10° C. above the biosynthesis bath temperature and acidification of the media can also improve bacterial cell yields by agglomeration prior to separation by centrifugation.

Drum drying of the sedimented bacteria is an acceptable method for preparing bacterial biomass for protein supplement (Gutcho, 1973, above). Part of the water is removed and an oil-enriched product can be removed from above the nip of the rollers and a microorganism enriched product removed just below the nip. The bacterial material is flaky which aids in the following solvent extraction step and recovery of the bacterial biomass by filtration.

Both the processes of cell recovery involve complex procedures of purification and show a very expensive method of both fermentation and recovery of cells, which makes it mote difficult for any consumer application.

The process of the present invention avoids these complex recovery steps.

Method of biomass Recovery According to the Present Invention.

The process of the present invention provides a simplified process for fermentation, recovery of cells and isolation of other value added products. The solvent extraction and separation of the cells from the bulk is achieved in a very simple manner. The bacterial biomass will either be in a dried state through drum drying or the form of a slurry, which may be spray dried and preserved for extended periods for incorporation in the feed as a supplementary nutrient.

TABLE 1
Chemical Analysis of SpillRemed Biomass
The biomass produced during fermentation of waste oils
and other hydrocarbon and vegetable oils should approximate
that of the SpillRemed consortium of bacteria which were
used as a control, and were analyzed for the chemical
application of the biomass in industry.
Analysis of chemical constituents
Whole Cells (Wet wt: 2.5 gms/liter)Cell free extract

| | | |
|---|---|---|
| Carb.: | 24.45% | 55.99 |
| Lipids | 8.12% | 0.73 |
| Proteins | 34.20 | 43.27 |
| DNA | 0.42 ug | 0.0 |
| (LPS) | 22.29% | 0.0 |

Chemical composition:
Amino acid values of cells from biomass:

| | | |
|---|---|---|
| Asp | 11.16 | 14.65 |
| Glu | 11.55 | 9.8 |
| Seri | 4.81 | 6.46 |
| His | 1.12 | 1.59 |
| Gly | 5.18 | 4.97 |
| Thr | 6.16 | 9.01 |
| Ala | 9.1 | 9.88 |
| Arg | 6.09 | 3.43 |
| Tyr | 5.2 | 3.31 |
| Val | 6.45 | 6.64 |
| Met | 1.84 | 0.91 |
| Phe | 5.66 | 4.78 |
| Ile | 5.27 | 3.87 |
| Leu | 9.05 | 7.01 |
| Lys | 8.13 | 8.54 |
| Pro | 3.22 | 5.18 |

Evaluation as Feed

Testing of SCP with animals has been done by a limited number of workers but available literature indicates that single cell protein is not rejected by cattle. In a recent paper, studies were carried out in Colorado State University to determine the following a) Palatability of SCP b) Comparative evaluation of SCP to conventional protein supplement and c) to compare carcass characteristics of cattle fed with SCP and conventional protein supplement. The results showed that the steers readily consumed single cell protein and the animal performance was comparable with the conventional feed (Phetteplace et al., 2000). The biomass produced through the process described in the proposed patent is thus considered safe for animal feed. It may also be used as supplemental addition to human food if needed.

Innovativeness and Originality of the Process from Prior Art

The process that we propose differentiates itself from the earlier work in the following aspects:

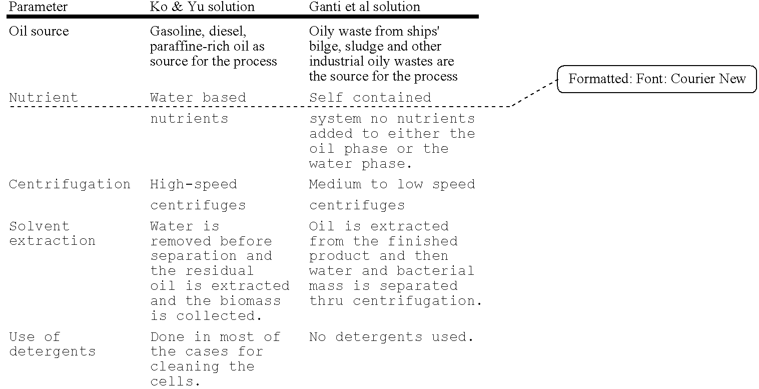

The results indicate that the bacterial biomass is very rich in proteins and carbohydrates both in the cell and in the extra-cellular polymeric substances (EPS).

The results obtained from analysis of ECP are remarkable in that the substance is rich in protein and carbohydrate and does not contain any endotoxin (LPS) and should be considered as safe for any nutritive addition.

The nature of substance has earlier been classified as biosurfactants and the nature of ECP in recent studies reflect the possibilities of being potential biosurfactants. It is believed that the signature analysis of the ECP will vary from waste oil to waste oils in general and composition of the microbial composition in particular. The process described above can be carried out with a consortium of bacteria or single species of bacteria, yeasts and fungi. In view of the fact that the microbes are being utilized for biomass production and not released in nature even genetically modified species can be employed without any concern for environment.

There has thus been shown and described a novel process for the production of biomass, including surfactants and Single Cell Protein from industrial waste oils, oily sludge from ships and other sources of oily wastes which fulfills all the objects and advantages sought therefore. Many changes, modifications, variations and other uses and applications of the subject invention will, however, become apparent to those skilled in the art after considering this specification which discloses the preferred embodiments thereof. All such changes, modifications, variations and other uses and applications which do not depart from the spirit and scope of the invention are deemed to be covered by the invention, which is to be limited only by the claims which follow.

1. A method of producing whole cell biomass and biosurfactants from waste oil through bioremediation, comprising the steps of:
A) Isolating a source of waste oil in a container,
B) providing an oleophilic suspension comprising physiologically active, species/strains of bacteria that are oil-consuming, in a fatty substance, comprising an oleophilic nutrient as a source of nitrogen and phosphorus for said bacteria, and admixing it with the source of waste oil and an excess of water,
C) Agitating the mixture of waste oil, oleophilic suspension and water for a fixed period of time, to produce biomass, carbon dioxide, and water by the consumption of oil by the bacteria;
D) Removing the residual oil by solvent extraction;
E) Centrifuging the remaining mixture at a low to medium speed of less than about 20,00 rpm, to separate the whole cells, from the cell free, oil-free, water-based liquor
F) Filtering the liquor through 0.22 micron filter paper.
G) Collection of cell free nutrient rich filtrate
H) Precipitation of a bio-surfactant, extra cellular polymeric substance from filtrate produced in step F by adding a solvent selected from the group consisting of:
water mixable acetone, alcohol and inorganic salts to the filtrate; storing the mixture for at least about 10 hours at 4° C. for precipitation, and
I) Centrifuging at a speed less than about 20,000 rpm, to separate the precipitate.

2. A nutritional supplement comprising protein isolated from the biomass produced by the method of claim 1

3. A nutritional supplement isolated from the cell free extract produced by the method of claim 1.

4. The method of claim 1, wherein the consortium of oil consuming bacteria comprise bacteria selected from the group consisting of: Pseudomonas aeruginosa, Phenylobacterium immobile, Stenotrophomonas maltophila, Gluconobacter cerius, Agrobacterium radiobacter and Citrobacter freundii and other indigenously available species

5 The method of claim 1, wherein the container is a fermentor.

6. The method of claim 1, wherein the bacteria is genetically modified.

7. A method of producing vitamins, antibiotics and tailor made bio-surfactant according to the method of claim 1, wherein the bacteria are pure strains of bacteria or yeasts.

8. A method of producing vitamins, antibiotics and tailor made bio-surfactant according to the method of claim 1, wherein the bacteria are genetically modified bacterial or yeast species.

9. A continuous batch method, comprising the method of claim 1, wherein the solvent and oil extracted in step D are separated, the solvent recycled, and the residual oil recycled to the container.

10. The method of claim 1, wherein agitation of the admixture supplies oxygen from the atmosphere to the admixture, to produce biomass.

11. A biosurfactant obtained by solvent precipitation of the cell free extract produced by the method of claim 1.

12. A nutritional supplement comprising the extra cellular polymeric substance produced by the method of claim 9.

13. An economical method of producing whole cell biomass comprising about 24% by weight carbohydrate and about 34% by weight protein, from waste oil, comprising the steps of:
A) Isolating a source of waste oil in a container,
B) providing an oleophilic suspension comprising physiologically active, species/strains of bacteria that are oil-consuming, in a fatty substance, comprising an oleophilic nutrient as a source of nitrogen and phosphorus for said bacteria, and admixing it with the source of waste oil and an excess of water,
C) Agitating the mixture of waste oil, oleophilic suspension and water for a fixed period of time, to produce biomass, carbon dioxide, and water by the consumption of oil by the bacteria;
D) Removing the residual oil by solvent extraction; and;
E) Centrifuging the remaining mixture at a speed less than about 20,000 rpm, to separate the whole cells from the cell free, oil-free, water-based liquor

14. The method of claim 13, wherein the container is a fermentor.

15. A continuous batch method, comprising the method of claim 13, wherein the solvent and oil extracted in step D are separated, the solvent recycled, and the residual oil recycled to the container.

16. A method for eliminating waste oil, comprising the steps of:
A) Isolating a source of waste oil in a container,
B) providing an oleophilic suspension comprising physiologically active, species/strains of bacteria that are oil-consuming, in a fatty substance, comprising an oleophilic nutrient as a source of nitrogen and phosphorus for said bacteria, and admixing it with the source of waste oil and an excess of water,
C) Agitating the mixture of waste oil, oleophilic suspension and water for a fixed period of time, to produce biomass, carbon dioxide, and water by the consumption of oil by the bacteria;
D) Removing the residual oil by solvent extraction;

17. The method of claim 16, wherein the container is a fermentor.

18. A continuous batch method, comprising the method of claim 16, wherein the solvent and oil extracted in step D are separated, the solvent recycled, and the residual oil recycled to the container.
